(19) United States
(12) Patent Application Publication
(10) Pub. No.: US 2008/0071227 A1
(43) Pub. Date: Mar. 20, 2008

(54) DOSE SETTING MECHANISM FOR SETTING FINE DOSES

(76) Inventors: Ulrich Moser, Heimiswil (CH); Christian Schrul, Burgdorf (CH); Juerg Hirschel, Aarau (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/842,677

(22) Filed: Aug. 21, 2007

Related U.S. Application Data
(63) Continuation of application No. PCT/EP2006/001616, filed on Feb. 22, 2006.

(30) Foreign Application Priority Data
Feb. 22, 2005 (DE) 102005008065.0
May 24, 2005 (DE) 102005023821.1

(51) Int. Cl. A61M 5/315

(52) U.S. Cl. 604/207

(57) ABSTRACT

A dose setting mechanism for the dosed administration of an injectable substance from an injection device, the mechanism including a threaded rod, wherein the thread extends around the rod and carries teeth wherein neighboring teeth lie at a first distance from one another, a first rotation limiting element connected to the injection device and having at least one detent element lockable with the rod, and a second rotation limiting element connected to a rotary sleeve of the injection device and including at least one detent element lockable with the rod, wherein the second rotation limiting element is at a distance from the first rotation limiting element and the detent elements of the rotation limiting elements are arranged such that when the threaded rod is rotated in relation to at least one of the rotation limiting elements for less than the first distance, the detent elements lock in an intermediate position.

### CROSS-REFERENCE TO RELATED APPLICATIONS

This application is a continuation of International Application No. PCT/EP2006/001616 filed on Feb. 22, 2006, which claims priority to German Application No. DE 10 2005 008 065.0 filed on Feb. 22, 2005 and German Application No. DE 10 2005 023 821.1 filed on May 24, 2005, the contents of all of which are incorporated in their entirety herein by reference.

### BACKGROUND

The present invention relates to devices for injecting, infusing, delivering, dispensing or administering substances, and to methods of making and using such devices. More particularly, the present invention relates to a dose setting or selecting device or mechanism for injection devices for administering set or selected doses of an injectable substance or product and, more particularly, to a dose setting mechanism comprising a threaded rod with a toothed thread, whereby fine, small and/or precise doses of an injectable product may be set or selected and injected.

U.S. Pat. No. 6,325,004 relates to a syringe for administering settable doses of a medicament from a cartridge, with a housing which contains a holder for holding the cartridge containing medicament, a plunger rod with a non-circular cross-section and an external thread, a plunger rod drive, a one-way coupling with a crown-shaped ring of inner locking grooves, the crown shaped ring of which can be integrated in the housing, and a lock with at least one pair of resilient arms, and each of the arms has a free end.

Other known dose setting mechanisms also include a threaded rod, some with a toothed rod with a large number of closely lying teeth or a set of teeth for setting injectable doses. However, such mechanisms usually require threaded rods with shorter distances between the teeth, and such rods are technically very complex to manufacture.

### SUMMARY

One object of the present invention is to overcome the disadvantages known from the prior art. Another object is to provide a dose setting mechanism for an injection device that is easy to manufacture, by which smaller or finer doses of an injectable product can be set and administered.

In one embodiment, the present invention comprises an injection device, a dose setting mechanism associated or incorporated with the injection device, and a method of setting a dose to be injected by the injection device. In one embodiment, the injection device and/or the dose setting mechanism comprise a rod or rod-like member carrying an array, set or sets of teeth.

In one embodiment, the present invention comprises a dose setting mechanism for the dosed administration of an injectable substance from an injection device, the mechanism including a threaded rod, wherein the thread extends around the rod and carries teeth wherein neighboring teeth lie at a first distance from one another, a first rotation limiting element connected to the injection device and having at least one detent element lockable with the rod, and a second rotation limiting element connected to a rotary sleeve of the injection device and including at least one detent element lockable with the rod, wherein the second rotation limiting element is at a distance from the first rotation limiting element and the detent elements of the rotation limiting elements are arranged such that when the threaded rod is rotated in relation to at least one of the rotation limiting elements for less than the first distance, the detent elements lock, in one embodiment, in an intermediate position.

In one embodiment, the present invention comprises a dosing device and an injection device for the dosed administration of an injectable product from the injection device, the dosing device comprising a threaded rod, wherein the thread runs around its periphery and carries teeth, including neighboring teeth which run or extend along the thread in the peripheral direction of the threaded rod and lie at a first distance from one another in said peripheral direction. The dosing device further comprises a first rotation limiting element connected to the injection device or a housing part of the injection device and comprising at least one detent element that locks with the threaded rod, and a second rotation limiting element connected to a rotary sleeve of the injection device and comprising at least one detent element that locks with the threaded rod, wherein the second rotation limiting element is at a distance from the first rotation limiting element and the detent elements of the rotation limiting elements are arranged and/or configured such that, when the threaded rod is rotated in relation to at least one of the rotation limiting elements for less than the first distance, the detent elements lock in an intermediate position.

In one embodiment, a dose setting mechanism in accordance with the present invention is used in an injection device for administering set doses of an injectable product, which enables the finest doses possible to be set and then administered. Such an injection device has a housing or housing part in which a setting sleeve for setting a one-off initial dose, for adjusting the injection device or for setting several different dose quantities is rotatably mounted. Inside such a setting sleeve, a rotating sleeve can be rotatably mounted, which has an external thread which is able to engage in an internal thread of the injection device or the housing part of the injection device. A dose quantity can be set by turning or rotating a setting knob or the like, which is connected to the rotating sleeve. By turning the knob to set or select a dose quantity, the knob is moved together with the rotating sleeve relative to the setting sleeve in a direction opposite that in which the injectable product is dispensed. Furthermore, a dose quantity can firstly be set by rotating the setting sleeve and then primed by pulling the knob so that the knob and sleeve are moved opposite the direction in which the injectable product is dispensed. To dispense a set or selected dose, the knob can be pushed in the dispensing direction, as a result of which the rotating sleeve is screwed into the injection device causing a threaded rod to be moved in rotation. The rod is able to push a stopper disposed against the front face of the rod into an ampoule to compress a substance contained in the ampoule and thus dispense the set dose.

In one embodiment of the present invention, the dose setting mechanism for administering set doses of an injectable product from an injection device comprises a threaded rod with a thread extending around it on the outside surface. The thread carries teeth, or teeth are formed on the thread. The teeth or teeth includes a plurality of individual teeth, which, in some embodiments, are disposed along the thread pitch of the thread at a constant first distance from one another in the circumferential direction. Several threads, each with teeth or a set of teeth, may also may provided on the threaded rod, in which case the teeth of a thread are disposed at a constant first distance from one another along the respective thread of the threaded rod in the circumferential direction.

In some embodiments, the dose setting mechanism further comprises a first rotation limiting element, which is fixedly connected to the injection device or a housing part of the injection device. The rotation limiting element has at least one, e.g. one, two, three, four or more, catch element. The catch element(s) are designed to be able to latch with the teeth of the threaded rod. If one or more catch elements latch with the teeth, the threaded rod can be rotated or screwed in relative to the catch element(s), in some preferred embodiments in one direction only, for example in a clockwise or anti-clockwise direction, so that it is rotated in the direction towards the distal end of the injection device, because a rotation in another direction is prevented due to a force of the catch element transmitted to the threaded rod or because the threaded rod is retained by the catch element.

In some preferred embodiments, the teeth associated with the threaded rod are in the form of non-elastic sawtooth cams (e.g., a series, sequence or progression of peaks, tips or raised portions, which may be rounded or suitably shaped, separated by valleys or relieved areas, which also may be suitable shaped). The teeth or cams may have a contact side extending perpendicular to the threaded rod and a sliding side disposed obliquely with respect to the threaded rod. When a catch element latches with such cams, the threaded rod can be rotated in one direction only, the direction in which the catch element is able to slide along the oblique sliding face, in which case the catch elements of the rotation limiting elements, which may be radially displaceable relative to the threaded rod, are able to effect a radial movement accordingly or along the pitch of the sliding side. A rotation in the opposite direction is not possible when a catch connection or latching action is effected because the catch element slides on the perpendicular contact side of the cam and is therefore able to expend a force opposing the intended rotation or rotation direction, thereby preventing the rotation and holding the rod secure.

In some embodiments, the dose setting mechanism in accordance with the present invention further comprises a second rotation limiting element fixedly connected to the rotating sleeve of the injection device, which has at least one catch element and, in some preferred embodiments, one, two, three, four or more catch elements. In some preferred embodiments, the second rotation limiting element is at a distance apart from the first rotation limiting element in the longitudinal direction or along the longitudinal axis of the rod and/or injection device.

In some preferred embodiments, the first and the second rotation limiting elements each comprise two or three catch elements. The catch elements of the second rotation limiting element, no matter how many there are, may be of exactly the same design as the catch elements of the first rotation limiting element or may be different. The at least one catch element of the second rotation limiting element is designed so that it permits a latching with the teeth of the threaded rod, as described above.

A dose quantity can be set by rotating or pulling a dose setting knob connected to or coupled with the rotating sleeve, whereby the second rotation limiting element connected to the rotating sleeve is moved relative to the threaded rod in the proximal or rear direction opposite the dispensing direction of the injectable product, while the threaded rod is held stationary due to the latching action with the first rotation limiting element. When the dose setting knob is depressed, the threaded rod guides a rotating movement and a translating movement relative to the first rotation limited element caused by a force transmitted by the dose setting knob to the rotating sleeve and by a force transmitted by the second rotation limiting element connected to the rotating sleeve to the threaded rod. This results in the set dose of the injectable product being dispensed.

To set fine doses, in some embodiments the at least one catch element of the first or second rotation limiting element is disposed or designed so that when a dose quantity is set, which is done by rotating at least one of the rotation limiting elements, e.g., the second rotation limiting element, relative to the threaded rod by less than the first distance between the teeth of the teeth in the circumferential direction of the threaded rod, the at least one catch element is able to latch in an intermediate position. The distance of the intermediate position from the initial position or the rotation path or path covered before reaching the intermediate position of at least one or more of the rotation limiting elements relative to the threaded rod is shorter than the first distance between the teeth in the circumferential direction. In the case of conventional dose setting mechanisms, a rotating movement which is shorter than the distance between two adjacent teeth would result in the threaded rod sliding back into the original position or initial position and a dose would therefore not be set. A minimum dose could only be set by a rotation path corresponding to at least the distance between two adjacent teeth in the case of conventional dose setting mechanisms. In the case of the dose setting mechanism in accordance with the present invention, smaller doses can be obtained or set by setting intermediate positions which are not as far apart.

The teeth disposed adjacent to one another in the longitudinal direction of the threaded rod, in other words the teeth extending along the longitudinal axis on a line extending parallel with the longitudinal axis or on a zigzag line extending along the longitudinal axis, may be disposed in a same position in the circumferential direction of the threaded rod so that the teeth lie along the longitudinal axis on a line of the threaded rod parallel with the longitudinal axis of the threaded rod. In some embodiments, the teeth adjacent to one another longitudinally are offset from one another in the circumferential direction at an angle to the longitudinal axis of the threaded rod, for example, so that there are no directly adjacent teeth in the longitudinal direction which extend along a line parallel with the longitudinal axis. In some preferred embodiments, the distance in the circumferential direction of the adjacent teeth disposed obliquely in the longitudinal direction corresponds to half the first distance between teeth disposed in a same position in the longitudinal direction. However, this distance may also be a third or a quarter of the first distance. If, for example, n (n ε N) catch elements are provided on one or both rotation limiting elements, the adjacent teeth extending at an angle to the longitudinal axis of the threaded rod are disposed at a distance from one another in the circumferential direction which corresponds to the n-th fraction of the first distance between the teeth disposed adjacent to one another at a same position and in the circumferential direction. By providing adjacent teeth at an angle in the longitudinal direction of the threaded rod or along its length, and based on an expedient choice of the rotation limiting elements or catch elements or with an appropriate design or appropriate disposition of the rotation limiting elements or catch elements, fine or finer dose quantities can be set.

In some preferred embodiments, one or both of the rotation limiting elements may have two or more catch elements which are disposed along the circumference of the threaded rod and can engage in the teeth. For example, two catch elements may lie virtually opposite one another so that the deviation from a precise or specific oppositely lying position may be described by an offset. The two catch elements may be disposed at a distance from one another along the circumference which corresponds to virtually half the circumference of the threaded rod. The deviation from the exact half circumference of the threaded rod may be described by an offset of the catch elements from one another. In particular, the engaging tips of the catch elements which lie directly on the surface or circumference of the threaded rod when the catch elements latch with the teeth may be disposed at such a distance or offset from one another. The offset between the catch elements is therefore half of the first distance between the teeth. Three catch elements may also be disposed along the circumference of the threaded rod, which are virtually disposed at a distance from one another which corresponds to a third of the circumference of the threaded rod. The respective offset of the three catch elements from the exact one third is thus one third of the distance of the teeth from one another. If n catch elements are disposed on a rotation limiting element around the circumference of the threaded rod for example, the offset of the adjacent catch elements from one another in the circumferential direction of the threaded rod is the n-th fraction of the first distance between the teeth. Due to the offset of the catch elements from one another, the catch elements may latch alternately with the teeth to ensure a dose setting or dispense a dose. Due to the alternating latching arrangement, smaller dose quantities can be set and dispensed. If the offset in the circumferential direction is half the first distance between the teeth, a dose can be set which is half as big as it would be without such an offset.

In some embodiments, a catch element or several catch elements may have a plurality of engaging elements rather than a single engaging tip, for example two, three, four or more engaging elements, which may be disposed at a constant distance from one another. If using two engaging elements per catch element, the distance (which may be referred to as the engaging element distance) may be half the first distance of the teeth from one another, for example, or if using three engaging elements per catch element may correspond to a third of the first distance. If n engaging elements are disposed on a catch element, for example, the distance between the engaging elements is the n-th fraction of the first distance between the teeth. By providing two or more engaging elements on one or more catch elements, finer doses can be set with the dose setting mechanism and administered with the injection device as described above in connection with the offset of the catch elements from one another.

In some preferred embodiments, two or more teeth in the longitudinal direction of the threaded rod may be disposed at a second distance from one another in the longitudinal direction, or all the adjacent teeth along the longitudinal axis of the threaded rod may be disposed at a second constant distance from one another. The catch elements of a rotation limiting element or both rotation limiting elements may be disposed at a same position in the longitudinal direction of the threaded rod or may be spaced apart from one another in the longitudinal direction of the threaded rod. In some preferred embodiments, the distance between the catch elements of a rotation limiting element in the longitudinal direction corresponds to the second distance of the adjacent teeth from one another in the longitudinal direction so that the teeth can be guided along the catch elements and the catch elements can latch or engage on each and every tooth.

In some embodiments, one catch element or several catch elements that are wider than a tooth and extend along two, three, four or more teeth along the longitudinal axis of the threaded rod may be provided. If the adjacent teeth in the longitudinal direction are disposed at a same position in the circumferential direction, for example, the adjacent teeth therefore extend in the longitudinal direction on a line parallel with the longitudinal axis of the threaded rod, and a catch element can extend along two, three, four or more teeth and latch with each of the teeth disposed on the parallel line. If the teeth of the teeth adjacent to one another in the circumferential direction are oblique or if the teeth adjacent to one another in the longitudinal direction are offset from one another in the circumferential direction, the wide catch element may extend along two, three or four teeth but latch with only the first or with the first and third tooth, for example. If the wide catch element is moved or rotated relative to the threaded rod to set or dispense a dose, for example, the wide catch element may latch with the second or with the second and fourth tooth and disengage from the other teeth so that smaller or finer rotating movements of the threaded rod can be effected relative to the catch element before latching again, which enables finer doses to be set.

Based on the different options for setting a finer or smaller dose, in accordance with the present invention, in a first state, such as an initial state of the dose setting mechanism, the contact side of a first catch element lies against a co-operating stop or stop side of a tooth to permit or prevent a rotation of the threaded rod. In this first state, the contact side of a second catch element of the same rotation limiting element lies not on a co-operating stop, but on a sliding side of a tooth of the teeth so that the threaded rod is able to move along the tooth. When the threaded rod has moved to a second state, for example after running or performing a priming operation, the contact side of the first catch element does not lie against a co-operating stop of a tooth, and instead, the contact side of the second catch element lies against a co-operating stop of a tooth.

In some embodiments. the first distance between the teeth in the circumferential direction is shorter than the second distance of the adjacent teeth in the longitudinal direction or relative to the longitudinal length of the threaded rod, in which case the second distance may also be shorter than the first distance.

In some embodiments, the teeth extend along the thread of the threaded rod along the entire length of the threaded rod. The teeth may also be provided intermittently, for example, in mutually adjoining or spaced apart portions, in which case the teeth, for example, the distance between the teeth from one another may be the same or different in the portions.

By virtue of another aspect of the invention, the dose setting mechanism for administering set doses of an injectable product from an injection device comprises a threaded rod which, instead of teeth, has a thread-shaped guide groove extending around its circumference, in which case a plurality of engaging elements is provided along the longitudinal axis of the threaded rod between the portions of the guide groove. In particular, the engaging elements are disposed on the threaded rod in such a way that an engaging element is disposed between every guide groove portion and the adjacent guide groove portion longitudinally. In particular, in some embodiments, the engaging elements may also be disposed on the threaded rod in such a way that they are disposed along the longitudinal axis of the threaded rod on a line parallel with the longitudinal axis of the threaded rod.

In some embodiments, the dose setting mechanism also has a first rotation limiting element connected to the injection device or a housing part of the injection device, which totally surrounds the threaded rod and which is cylindrical in shape or designed as an open cylinder on the internal face of which one or more guide elements and teeth are provided. The guide element or the guide elements are designed so that they enable the threaded rod to engage in the guide groove so that the rotation limiting element can be moved along the threaded rod or along the guide groove of the threaded rod and effect a rotating movement and a translating movement relative to the threaded rod. The teeth provided on the internal face of the rotation limiting element are designed or disposed so that they can latch with the engaging elements disposed on the threaded rod and transmit a force to the threaded rod, for example for retaining or moving the threaded rod. The teeth disposed in the same position in the longitudinal direction of the threaded rod adjacent to one another in the circumferential direction are disposed at a first distance from one another in the circumferential direction.

In some embodiments, the dose setting mechanism also has a second rotation limiting element which is connected to a rotating sleeve of the injection device and completely surrounds the threaded rod. In some embodiments, the second rotation limiting element connected to the rotating sleeve is exactly the same as the first rotation limiting element connected to the injection device and has one or more guide elements on the internal face which are able to engage in the guide groove rod as well as teeth on the internal face which are able to latch with the engaging elements of the threaded rod. The adjacent teeth of the teeth in the circumferential direction of the threaded rod are disposed at a first distance from one another.

To enable fine doses to be set, the teeth of the rotation limiting element are disposed or designed so that when the threaded rod is rotated relative to one or both rotation limiting elements by less than the first distance of the teeth from one another, at least one of the engaging elements of the threaded rod is able to latch with the teeth in a second position. The distance covered to the point of latching in the second position is therefore shorter than the distance covered to the point of latching between teeth disposed in the same position in the longitudinal direction that are adjacent to one another in the circumferential direction. This enables finer doses to be achieved than is the case with conventional dose setting mechanisms, which permit latching between adjacent teeth in the circumferential direction only.

The teeth (which may also be in the form of suitably shaped texturing or contouring) on the internal face of the first and second rotation limiting elements may comprise two, three, four, five, six or more sets of part-teeth disposed offset from one another in the longitudinal direction of the rotation limiting element. The adjacent teeth of the adjacent part-teeth in the longitudinal direction may be disposed offset from one another in the circumferential direction of the rotation limiting element and may be disposed at a distance from one another corresponding to a half, a third, a quarter, a fifth or a sixth of the first distance between two adjacent teeth in the circumferential direction, which are disposed at a same position in the longitudinal direction of the rotation limiting element. It would also be possible to provide n sets of part-teeth on the rotation limiting element in the longitudinal direction of the rotation limiting element. The adjacent teeth in the longitudinal direction of the rotation limiting element of adjacent sets of part-teeth may be spaced apart from one another by the n-th fraction of the first distance in the circumferential direction.

The teeth may be provided on the internal face of the rotation limiting element across the entire length of the rotation limiting element or may be disposed intermittently. For example, the teeth may be disposed in a first portion along the longitudinal axis of the rotation limiting element at a constant distance from one another in the circumferential direction of the rotation limiting element and, in an adjacent or adjoining second portion along the longitudinal axis of the rotation limiting element, may be disposed at a constant distance in the circumferential direction of the rotation limiting element that is different from the distance in the first portion.

### BRIEF DESCRIPTION OF THE DRAWINGS

FIG. 1a is a detail of one exemplary embodiment of the dose setting mechanism in accordance with the present invention;

FIG. 1b is a front view of the embodiment of FIG. 1a;

FIG. 1b is a side view of the threaded rod of the embodiment of FIG. 1a;

FIG. 1d is another view of the threaded rod of the embodiment of FIG. 1a;

FIG. 1e is a view of a rotation limiting element of the embodiment of FIG. 1a;

FIG. 1f is another view of the rotation limiting element of the embodiment of FIG. 1a;

FIG. 2a is a detail of another embodiment of the dose setting mechanism of the present invention;

FIG. 2b is a front view of the embodiment of FIG. 2a;

FIG. 2c is a side view of the threaded rod of the embodiment of FIG. 2a;

FIG. 2d is another view of the threaded rod of the embodiment of FIG. 2a;

FIG. 2e is a view of a rotation limiting element of the embodiment of FIG. 2a;

FIG. 2f is another view of the rotation limiting element of the embodiment of FIG. 2a;

FIG. 3a is a detail of another embodiment of the dose setting mechanism in accordance with the present invention;

FIG. 3b is a front view of the embodiment FIG. 3a;

FIG. 3c is a side view of the threaded rod of the embodiment of FIG. 3a;

FIG. 3d is another view of the threaded rod of the embodiment of FIG. 3a;

FIG. 3e is a view of a rotation limiting element of the embodiment of FIG. 3a;

FIG. 3f is another view of the rotation limiting element of the embodiment of the dose setting mechanism in accordance with the present invention;

FIG. 4a is a detail of another embodiment of the dose setting mechanism in accordance with the present invention;

FIG. 4b is a detail of the embodiment of FIG. 4a, with the rotation limiting element open;

FIG. 4c is a detail of the embodiment of FIG. 4a, with the rotation limiting element open;

FIG. 4d is a view of the threaded rod of the embodiment of FIG. 4a;

FIG. 4e is a view of an open rotation limiting element of the embodiment of FIG. 4a;

FIG. 4f is another view of the rotation limiting element of the embodiment of FIG. 4a;

FIG. 5a depicts one embodiment of an injection device in accordance with the present invention including one embodiment of a dose setting mechanism in accordance with the present invention with a rotating sleeve fully rotated into the injection device;

FIG. 5b is a cross-section of the injection device illustrated in FIG. 5a; and

FIG. 5c is a cross-section of the injection device from FIG. 5a with a rotating sleeve primed.

### DETAILED DESCRIPTION

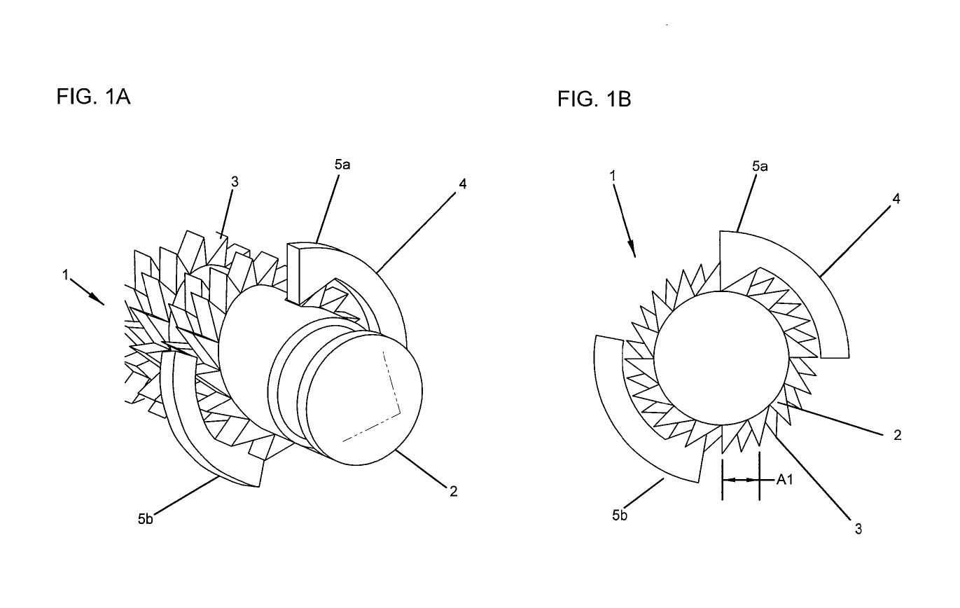

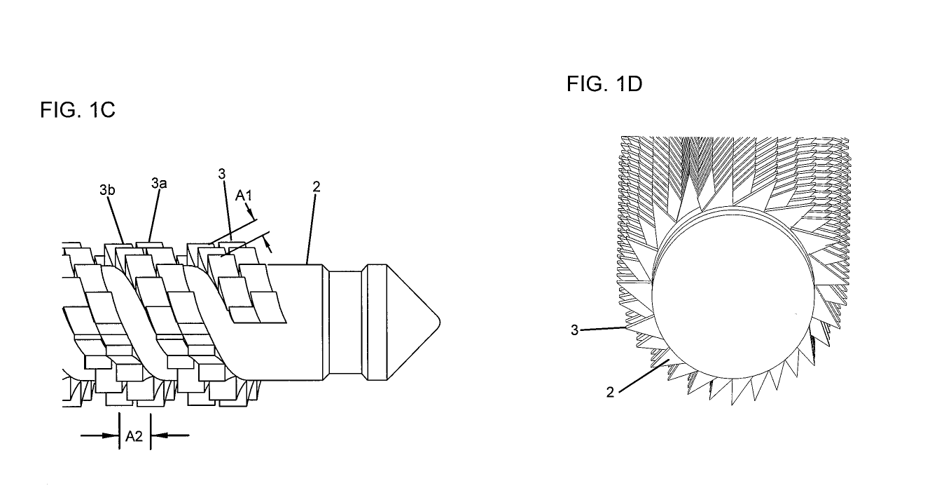

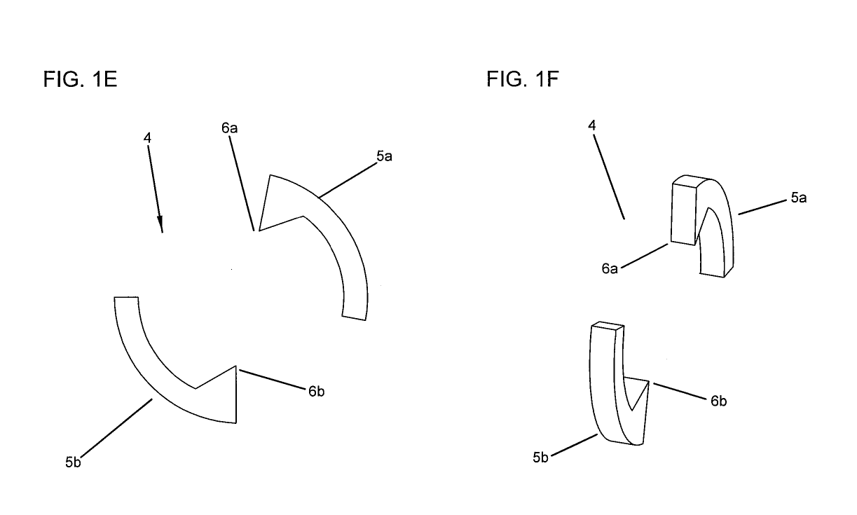

FIGS. 1a-1f show details and parts of a dose setting mechanism 1 with a threaded rod 2 in accordance with one embodiment of the present invention. The threaded rod 2 has a thread with teeth 3, the thread extending in the circumferential direction (i.e., extending around or circumferentially relative to) of the threaded rod 2, and two respective individual teeth of the teeth 3 lie adjoining or adjacent to one another in the longitudinal direction (along the longitudinal length) as may be seen from FIG. 1c. This embodiment may also be construed such that two mutually adjoining or mutually adjacent sets of teeth 3a, 3b are borne by one or more threads and extend around the threaded rod 2 generally on the surface thereof. Accordingly, the adjacent teeth of the teeth 3 in the longitudinal direction of the threaded rod 2 or the sets of teeth 3a, 3b adjoining one another in the longitudinal direction are offset from one another. The teeth adjoining one another in the longitudinal direction of the threaded rod 2 are disposed at a second distance A2 from one another in the longitudinal direction, and the adjacent teeth of the teeth 3 in the circumferential direction are disposed at a first distance A1 from one another. Around the threaded rod 2 and connected to an injection device or a housing part of the injection device is a rotation limiting element 4, which has two catch elements 5a, 5b offset from one another in the circumferential direction of the threaded rod 2. The catch elements 5a, 5b are also offset from one another in the longitudinal direction of the threaded rod 2, as may be seen from FIG. 1f, and this offset corresponds to the second distance A2 of the adjacent teeth in the longitudinal direction or the adjoining teeth of the teeth 3 in the longitudinal direction so that the teeth 3 are always guided along the catch elements 5a, 5b. In the circumferential direction of the threaded rod 2, the catch elements 5a, 5b, do not lie exactly opposite one another and the distance along the circumference of the threaded rod 2 between two engaging tips 6a, 6b of the catch elements 5a, 5b therefore does not exactly correspond to half the circumference of the threaded rod 2, or the imaginary extensions of the engaging tips 6a, 6b do not intersect at the center point or in the longitudinal axis of the threaded rod 2. The difference from the exact half circumference in terms of the distance between the catch elements 5a, 5b is described by an offset between the catch elements 5a, 5b. The offset between the catch elements 5a, 5b is exactly half of the first distance A1 in the circumferential direction between two teeth of the teeth 3 disposed at a same position in the longitudinal direction so that one catch element 5a is latched with the teeth 3, while the other catch element 5b is not latched with the teeth 3, as may be seen in FIG. 1b. When the catch elements 5a, 5b are rotated relative to the threaded rod 2 by an amount or a distance that is shorter than the first distance A1 between two adjacent teeth, for example to set a dose, the catch connection of the first catch element 5a with the teeth 3 is released, but the first catch element 5a does not latch again with an adjacent tooth spaced apart by the first distance A1 and instead, the second catch element 5b latches in an intermediate position with the teeth 3 so that the second catch element 5b is latched with a tooth adjacent to its initial position. When rotated again, the catch connection of the second catch element 5b with the teeth 3 is released and the first catch element 5a is already able to latch with a tooth in a position adjacent to its previous position after a distance that is shorter than the first distance A1. Consequently, smaller doses can be set than is the case with dose setting mechanisms 1 in which the smallest dose setting is obtained by a catch element 5a, 5b which is able to move respectively into an adjacent tooth in the circumferential direction.

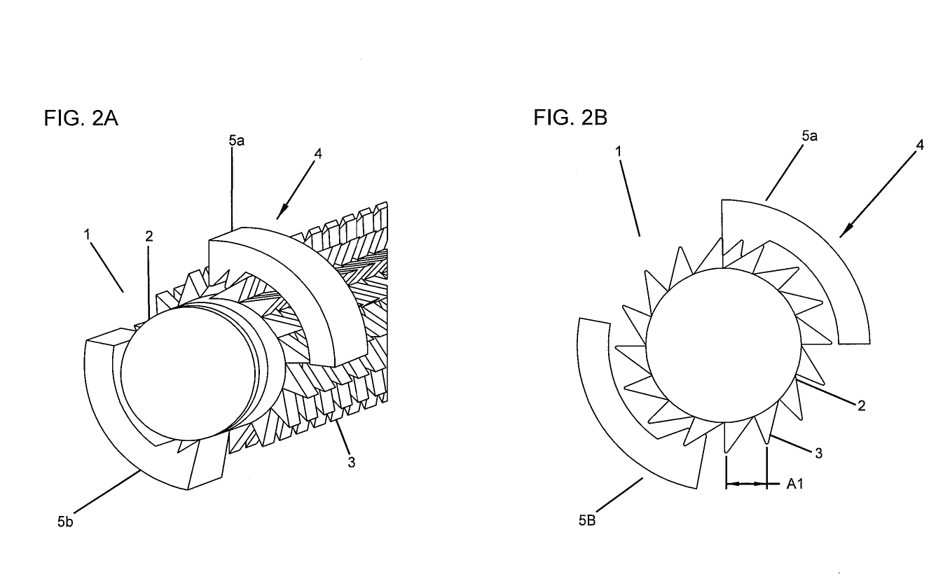

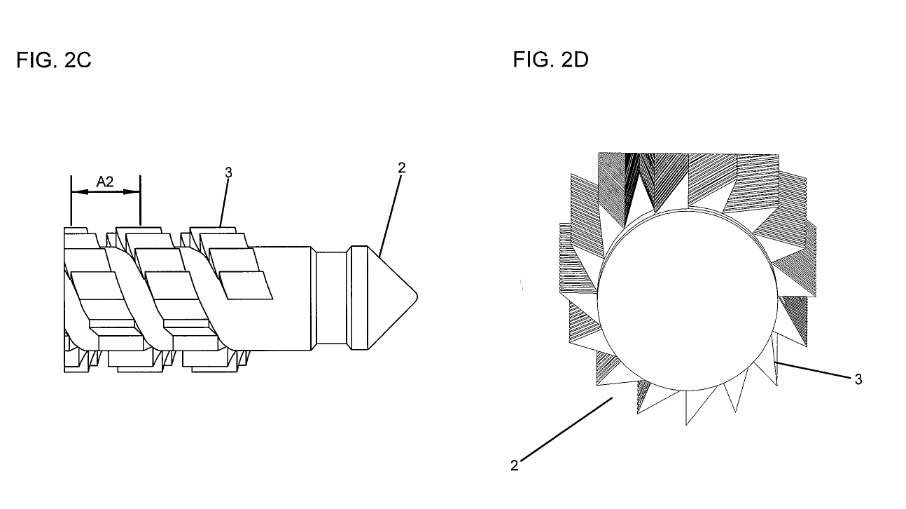

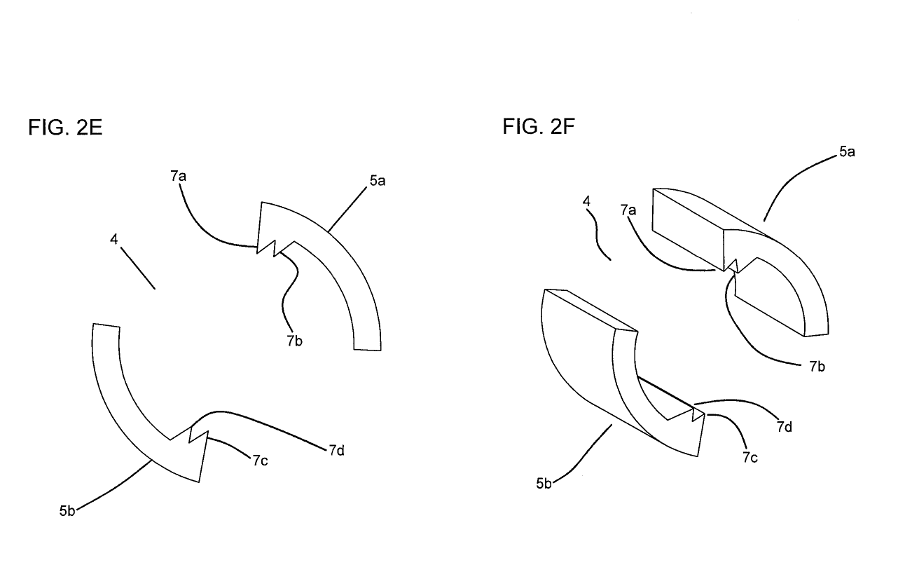

FIGS. 2a-2f show details and parts of a dose setting mechanism 1 with a threaded rod 2 in accordance with a second embodiment of the present invention, with teeth 3 extending uniformly around the threaded rod 2 along a thread. The teeth of the threaded rod 2 are disposed at a first constant distance A1 from one another in the circumferential direction and are disposed at a second constant distance A2 from one another in the longitudinal direction. The adjacent teeth in the longitudinal direction are also disposed in a same position in the circumferential direction of the threaded rod 2 or are not offset from one another. A finer dose can be obtained with this embodiment, among other things due to an offset of the catch elements 5a, 5b, as with the first embodiment, compared with an ideal oppositely lying position, which may be seen in FIG. 2e. Here too, the amount of the offset is half of the first distance A1 of the teeth from one another in the circumferential direction. Furthermore, a finer dose setting can be obtained due to the fact that two engaging elements 7a, 7b are provided on each of the catch elements 5a, 5b, the distance between them corresponding to half of the distance A1. When the rotation limiting element 4 is rotated out of a basic position illustrated in FIG. 2b, relative to the threaded rod 2 in the anti-clockwise direction by an amount that is shorter than the distance A1 of the teeth from one another in the circumferential direction, at least one of the catch elements 5a, 5b is already able to latch with the same or subsequent or adjacent tooth after a distance which corresponds to half of the distance A1 so that a dose can be set which is half as big as can be obtained with other dose setting mechanisms 1 where the teeth are disposed at the same distance A1 from one another in the circumferential direction.

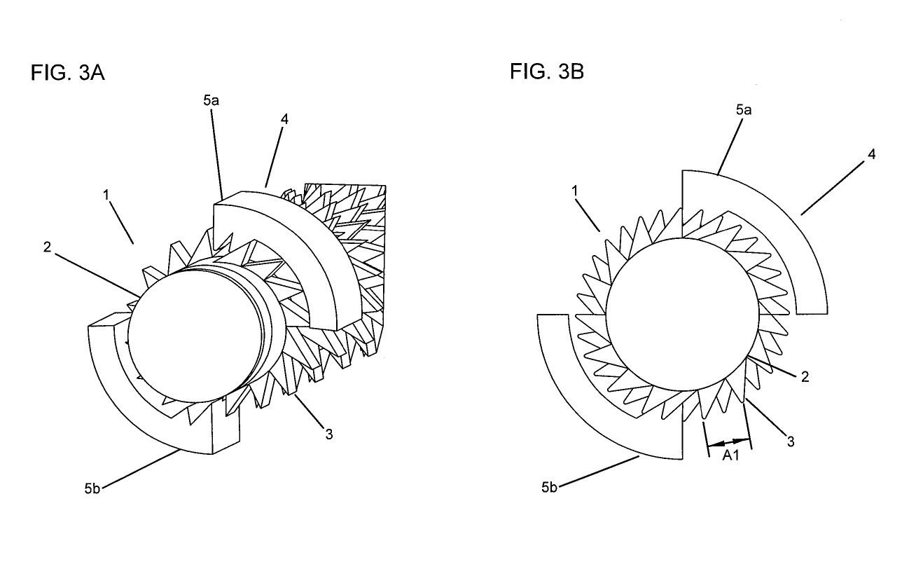

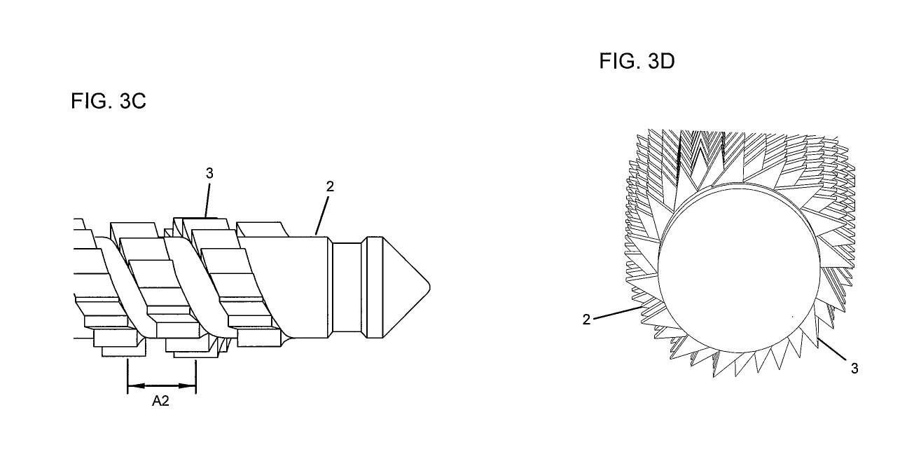

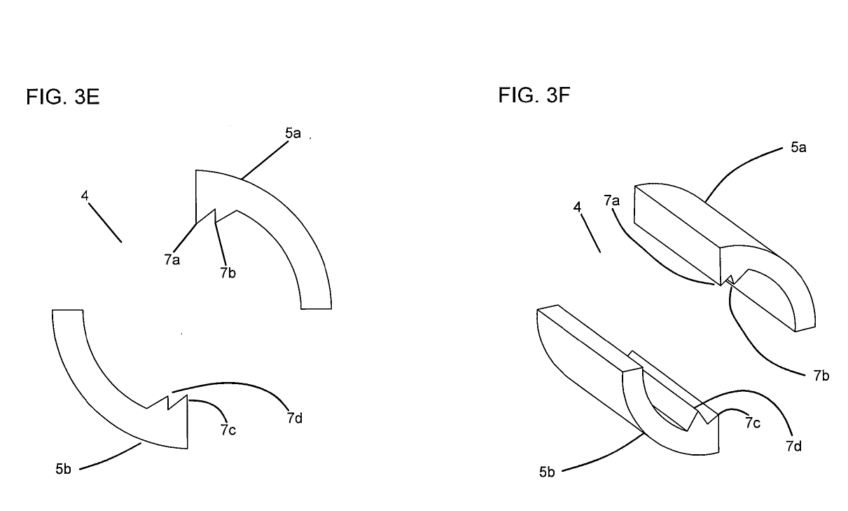

FIGS. 3a-3f show details and parts of a dose setting mechanism 1 with a threaded rod 2 in accordance with a third embodiment of the present invention, with teeth 3 provided in the circumferential direction of the threaded rod 2 along a thread on the threaded rod 2, and the adjacent teeth in the circumferential direction of the threaded rod 2 are disposed at a constant first distance A1 from one another and the adjacent teeth in the longitudinal direction, such as the obliquely adjacent teeth, are disposed at a second constant distance A2 apart from one another in the longitudinal direction of the threaded rod 2. The finer dose obtained with this embodiment is achieved, amongst other things, due to the fact that two teeth are disposed respectively offset from one another in the longitudinal direction of the threaded rod 2, in other words do not have the same position in the circumferential direction of the threaded rod 2. Furthermore, the two catch elements 5a, 5b have two engaging elements 7a, 7b which assist in setting a finer dose quantity. When one of the catch elements 5a, 5b latches with the teeth 3, as illustrated in FIG. 3b for example, the contact side of the catch element 5a extending perpendicular to the threaded rod 2 lies against a co-operating contact side of the tooth or cam extending perpendicular to the threaded rod 2, and the threaded rod can therefore not be rotated relative to the catch elements 5a, 5b in the clockwise direction or the catch elements 5a, 5b can not be rotated relative to the threaded rod in the anti-clockwise direction because this direction of movement is blocked by the catch element 5a latched with the teeth 3, for example, and the threaded rod 2 can be rotated relative to the catch elements 5a, 5b in the anti-clockwise direction only, or the catch elements 5a, 5b can be rotated relative to the threaded rod 2 in the clockwise direction only. When the catch elements 5a, 5b are rotated in the clockwise direction relative to the threaded rod 2 by an amount that is shorter than the distance A1 between two adjacent teeth in the circumferential direction, for example to set a dose, at least one of the catch elements 5a, 5b latches with the teeth again in an intermediate position before it has covered the distance corresponding to the distance A1. The distance covered in this particular embodiment corresponds to half of the first distance A1 but may also be one third, one quarter or another fraction of the first distance A1 depending on the disposition of the teeth 3 and the catch elements 5a, 5b. In the intermediate position, a tooth lies between the two engaging elements of at least one of the catch elements 5a, 5b or a contact side of at least one of the catch elements 5a, 5b lies against a co-operating stop of a tooth. Since, in order to set a smallest possible dose, it is possible to rotate the catch elements 5a, 5b relative to the threaded rod 2 by only a rotation corresponding to half the distance A1 between two teeth as far as another catch connection or another latch position, a dose twice as fine can be set or a dose quantity half the size can be set and dispensed using the dose setting mechanism 1.

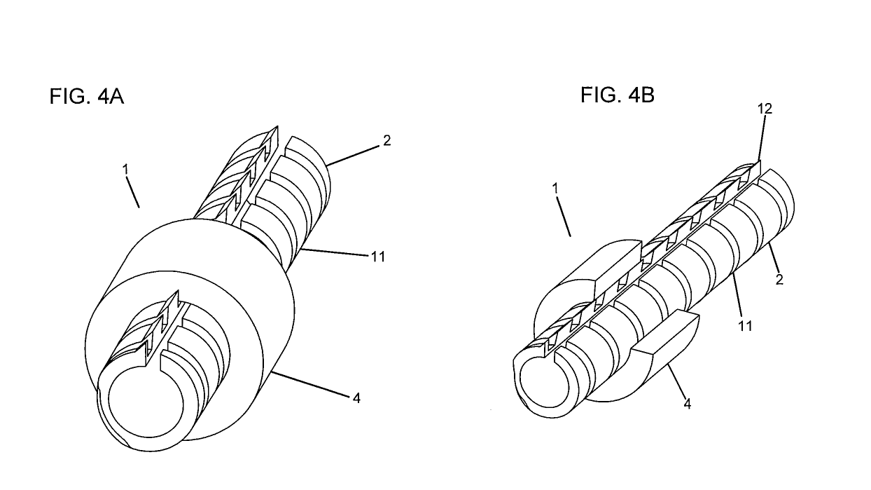

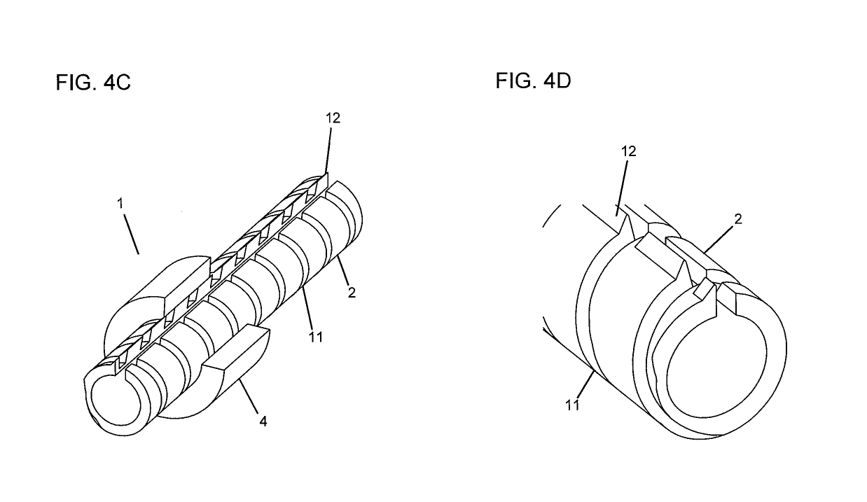

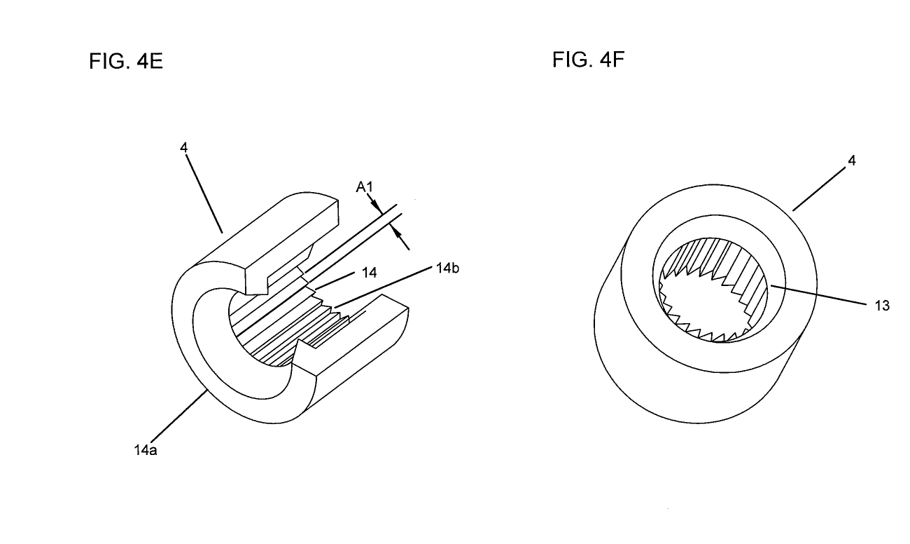

FIGS. 4a-4f illustrate details and parts of a dose setting mechanism in accordance with a fourth embodiment of the present invention, with a threaded rod 2 having a guide groove 11 extending round it and a plurality of engaging elements 12. The engaging elements 12 are disposed along a line parallel with the longitudinal axis of the threaded rod 2. The dose setting mechanism 1 also has a rotation limiting element 4, which may be connected to the injection device or to a rotating sleeve of the injection device, for example. The rotation limiting element 4 has teeth 14 and a guide element 13 on the internal face. The guide element 13 is designed so that it is able to engage in the guide groove 11 of the threaded rod and guided and rotated along the threaded rod 2, so that the rotation limiting element 4 is able to effect a relative rotating movement and a relative translating movement with respect to the threaded rod 2. The teeth 14 of the rotation limiting element 4 are designed so that it has a first part 14a and a second part 14b disposed offset from the first part 14a in the longitudinal direction of the rotation limiting element. The distances of the adjacent teeth of each set of part 14a, 14b from one another in the circumferential direction correspond to a first distance A1. The adjacent or obliquely adjacent teeth of the parts 14a, 14b in the longitudinal direction are disposed offset from one another in the circumferential direction of the rotation limiting element 4, and the offset corresponds to half of the first distance A1 between two adjacent teeth of a part 14a, 14b in the circumferential direction. The offset may also correspond to a third, a quarter or any other fraction of the first distance A1. As a result of this offset, the two parts or sets of teeth 14a, 14b are able to latch alternately with one of the engaging elements 12 of the threaded rod. Due to the alternating latching action, finer rotating movements can be effected in order to reach a catch or latch position than is the case with the situation of adjacent teeth of a part 14a, 14b in the circumferential direction latching with one of the engaging elements 12. Since the adjacent teeth in the longitudinal direction of the sets of teeth 14a, 14b are disposed at half the first distance A1 from one another in the circumferential direction, doses twice as fine can be set with the dose setting mechanism compared with using a conventional rotation limiting element.

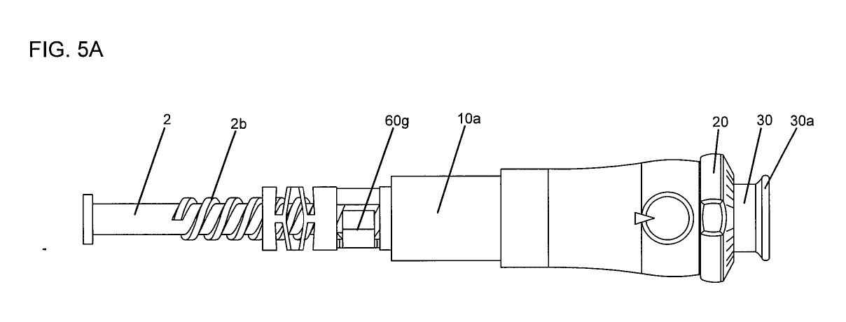

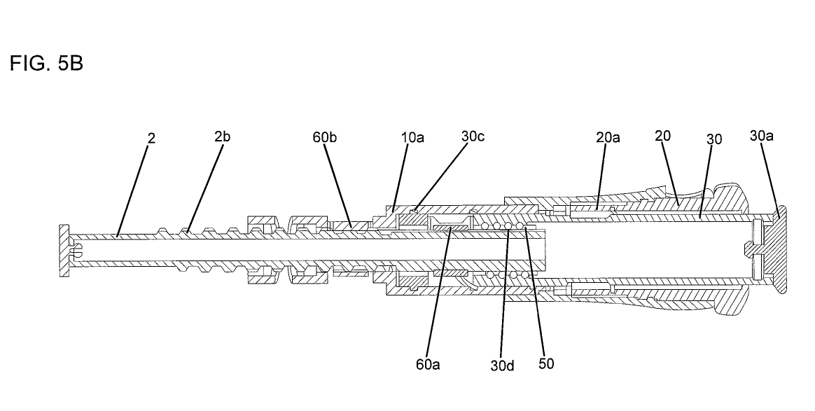

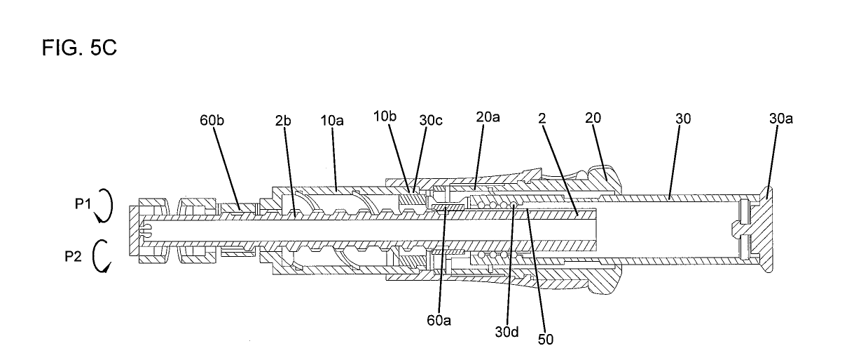

FIGS. 5a, 5b and 5c illustrate an injection device in accordance with the present invention incorporating one embodiment of a dose setting mechanism in accordance with the present invention. FIGS. 5a and 5b show a rotating sleeve 30 completely screwed into the injection device, which rotating sleeve 30 is guided by means of an external thread 30c in an internal thread 10b of the injection device housing 10a and has an internal thread 30d. When the injection device is primed by rotating the rotating sleeve 30 out, as illustrated in FIG. 5c, the threaded rod 2, which has a locking element 50 mounted on the threaded rod 2 so that it can not rotate but can slide axially, is retained by a rotation limiting element 60b connected to the injection device or to a housing part 10a of the injection device so that it can not rotate relative to the injection device housing 10a, while a rotation limiting element 60a connected to the rotating sleeve 30, which causes the rotating sleeve 30 to be rotated out of the housing 10a in the direction of rotation indicated by arrow P1, is not in a blocking engagement with the teeth on the thread 2b of the threaded rod 2. The rotation limiting element 60a has catch elements, which are mounted on arms 60g extending elastically around the threaded rod 2. When the rotating sleeve 30 mounted so as to be rotatable in a rotating knob 30a is rotated in the direction indicated by arrow P2 in FIG. 5c, e.g. by depressing the rotating knob 10a, the engaging elements of the rotation limiting element 60a of the rotating sleeve 30 engage with the teeth of the thread 2a of the threaded rod 2, so that as the rotating sleeve 30 is being screwed in, the threaded rod 2 is coupled with the rotating sleeve 30 and prevented from rotating and is driven and rotated with the latter. The rotating sleeve 30 can be screwed until a front or distal end of a setting sleeve 20, which has a setting lock cam 20a, lies against an internal stop of the injection device. The rotation limiting element 60b of the injection device permits a rotation of the threaded rod 2 relative to the injection device or the housing 10a, so that the threaded rod 2 is rotated by the rotating sleeve 30 and is guided in the internal thread of the injection device or housing 10a and is thus screwed out of the housing 10a in the distal direction in order to dispense a set and primed dose. The injection device can then be primed by extracting the rotating sleeve 30 in the direction indicated by arrow P1 in FIG. 5c.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A dose setting mechanism for administering set doses of an injectable product from an injection device, comprising:
a threaded rod with a thread bearing teeth extending in the circumferential direction, and adjacent teeth of the teeth along the thread in the circumferential direction of the threaded rod are disposed at a first distance from one another in the circumferential direction;
a first rotation limiting element connected to the injection device or to a housing part of the injection device, with at least one catch element for latching with the teeth of the threaded rod;
a second rotation limiting element connected to a rotating sleeve of the injection device with at least one catch element for latching with the teeth of the threaded rod; wherein
the at least one catch element of the rotation limiting element is designed or disposed so that when the threaded rod is rotated relative to at least one of the rotation limiting elements by less than the first distance, the at least one catch element is able to latch in an intermediate position.

2. The dose setting mechanism as claimed in claim 1, wherein adjacent teeth in the longitudinal direction of the threaded rod are disposed offset from one another in the circumferential direction by a distance corresponding to half of the first distance.

3. The dose setting mechanism as claimed in claim 1, wherein the at least one rotation limiting element has n catch elements and adjacent teeth in the longitudinal direction of the threaded rod are offset from one another in the circumferential direction by the n-th fraction of the first distance.

4. The dose setting mechanism as claimed in claim 1, wherein adjacent teeth in the longitudinal direction of the threaded rod are disposed in a same position in the circumferential direction.

5. The dose setting mechanism as claimed in claim 1, wherein at least one of the rotation limiting elements has two catch elements having engaging tips, wherein the engaging tips of the catch elements are spaced a distance from one another circumferentially relative to the rod and the distance is a predefined offset that is not a whole-number multiple of the first distance.

6. The dose setting mechanism as claimed in claim 1, wherein at least one of the rotation limiting elements has n catch elements having engaging tips, the tips being spaced a distance apart from one another around the rod, and the distance is a predefined offset that is not a whole-number multiple of the first distance and the offset corresponds to the n-th fraction of the first distance.

7. The dose setting mechanism as claimed in claim 1, wherein the at least one catch element has at least two or more engaging elements which are respectively disposed at a constant engaging element distance from one another.

8. The dose setting mechanism as claimed in claim 1, wherein the at least one catch element has n engaging elements respectively disposed at a constant engaging element distance from one another and the engaging element distance corresponds to the n-th fraction of the first distance.

9. The dose setting mechanism as claimed in claim 1, wherein at least two of the adjacent teeth of the teeth in the longitudinal direction of the threaded rod are disposed at a second distance apart from one another in the longitudinal direction.

10. The dose setting mechanism as claimed claim 9, wherein at least one of the rotation limiting elements has at least two catch elements and the catch elements are spaced apart in the longitudinal direction of the threaded rod by the second distance.

11. The dose setting mechanism as claimed in claim 1, wherein the catch elements are designed so that they extend in the longitudinal direction of the threaded rod along at least two teeth of the threaded rod.

12. The dose setting mechanism as claimed in claim 3, wherein the offset between the catch elements is such that when the dose setting mechanism is in a first state, the contact side of a first catch element is against a co-operating stop of a tooth and the contact side of a second catch element is not against a co-operating stop of a tooth, and in a second state, the contact side of the second catch element is against a co-operating stop of a tooth and the contact side of the first catch element is not against a co-operating stop of a tooth.

13. The dose setting mechanism as claimed in claim 1, wherein either the second distance is shorter than the first distance or the first distance is shorter than the second distance.

14. The dose setting mechanism as claimed in claim 1, wherein the rotation limiting elements can be moved radially relative to the threaded rod.

15. The dose setting mechanism as claimed in claim 1, wherein the teeth are respectively disposed at a constant distance from one another in the circumferential direction of the threaded rod in a first portion along the longitudinal axis of the threaded rod, and are respectively disposed at a different constant distance from one another in the circumferential direction of the threaded rod in a second adjacent portion along the longitudinal axis of the threaded rod, and the distance between the teeth in the first portion and the distance between the teeth in the second portion are different.

16. A dose setting mechanism for administering set doses of an injectable product from an injection device, comprising:
a threaded rod with a thread-shaped guide groove extending round it and a plurality of engaging elements disposed along a line parallel with the longitudinal axis of the threaded rod;
a first rotation limiting element connected to the injection device or a housing part of the injection device and surrounding the threaded rod, a guide element provided on the internal face of the rotation limiting element for engaging in the guide groove and teeth on the internal face of the rotation limiting element for latching with the engaging elements, adjacent teeth disposed at a same position in the circumferential direction of the rotation limiting element in the longitudinal direction of the first rotation limiting element are disposed at a first distance from one another in the circumferential direction;
a second rotation limiting element connected to a rotating sleeve of the injection device and surrounding the threaded rod, with a guide element disposed on the internal face of the rotation limiting element for engaging in the guide groove and teeth on the internal face of the rotation limiting element for latching with the engaging elements, the adjacent teeth disposed at a same position in the circumferential direction of the rotation limiting element in the longitudinal direction of the second rotation limiting element are disposed at a first distance from one another in the circumferential direction, wherein
the teeth of the rotation limiting elements are disposed so that when the threaded rod is rotated relative to at least one of the rotation limiting elements by less than the first distance, at least one of the engaging elements is able to latch with the teeth in an intermediate position.

17. The dose setting mechanism as claimed in claim 16, wherein the teeth comprise at least two sets of teeth offset from one another in the longitudinal direction of the rotation limiting element, and the adjacent teeth of the adjacent sets are disposed offset from one another in the circumferential direction of the rotation limiting element by a distance corresponding to one half or one third or one quarter or one fifth or one sixth of the first distance.

18. The dose setting mechanism as claimed in claim 17, wherein the teeth are across the entire length of the rotation limiting elements.

19. The dose setting mechanism as claimed in claim 17, wherein the teeth in a first tooth portion disposed along the longitudinal axis of the rotation limiting element are respectively disposed at a constant distance from one another in the circumferential direction of the rotation limiting element and in an adjacent second tooth portion along the longitudinal axis of the rotation limiting element are respectively disposed at a different constant distance in the circumferential direction of the rotation limiting element, and the distance between the teeth in the first portion and the distance between the teeth in the second portion being different.

20. An injection device comprising a dose setting mechanism comprising a rod carrying a circumferential array of teeth, adjacent teeth being at a first distance from one another, a first rotation limiting element operably coupled to the injection device and comprising at least one catch element for latching with the teeth, a second rotation limiting element operably coupled to a rotating sleeve of the injection device and comprising at least one catch element for latching with the teeth, wherein when the threaded rod is rotated relative to at least one of the rotation limiting elements by less than the first distance the at least one catch element latches in an intermediate position.

21. The injection device according to claim 20, wherein the rotation limiting elements have n catch elements and adjacent teeth are offset from one another in the by the n-th fraction of the first distance.
